(19) United States
(12) Patent Application Publication
(10) Pub. No.: US 2005/0090004 A1
(43) Pub. Date: Apr. 28, 2005

(54) Stem cell maturation for all tissue lines

(76) Inventor: Chauncey Bigelow Sayre, Irvine, CA (US)

Correspondence Address: Louis C. Cullman;Preston Gates & Ellis LLP, Suite 600, 1900 Main Street, Irvine, CA, 92614-7319, US

(21) Appl. No.: 10/864,788

(22) Filed: Jun. 8, 2004

Related U.S. Application Data
(63) Continuation-in-part of application No. 10/346,816, filed on Jan. 16, 2003.
(60) Provisional application No. 60/477,438, filed on Jun. 9, 2003.

(51) Int. Cl. C12N 5/08

(52) U.S. Cl. 435/366

(57) ABSTRACT

The present invention provides a hybrid stem comprising an enucleated adult stem cell having a nucleus derived from a primordial sex cell or an embryonic stem cell. The primordial sex cell may be a spermatogonia or an oogonia from a donor animal or mammal. The enucleated adult stem cell may be fused to a primordial sex cell or an embryonic stem cell by many methods including but not limited to electrofusion, virus-based fusion methodology, chemical fusion or mechanical-based fusion.

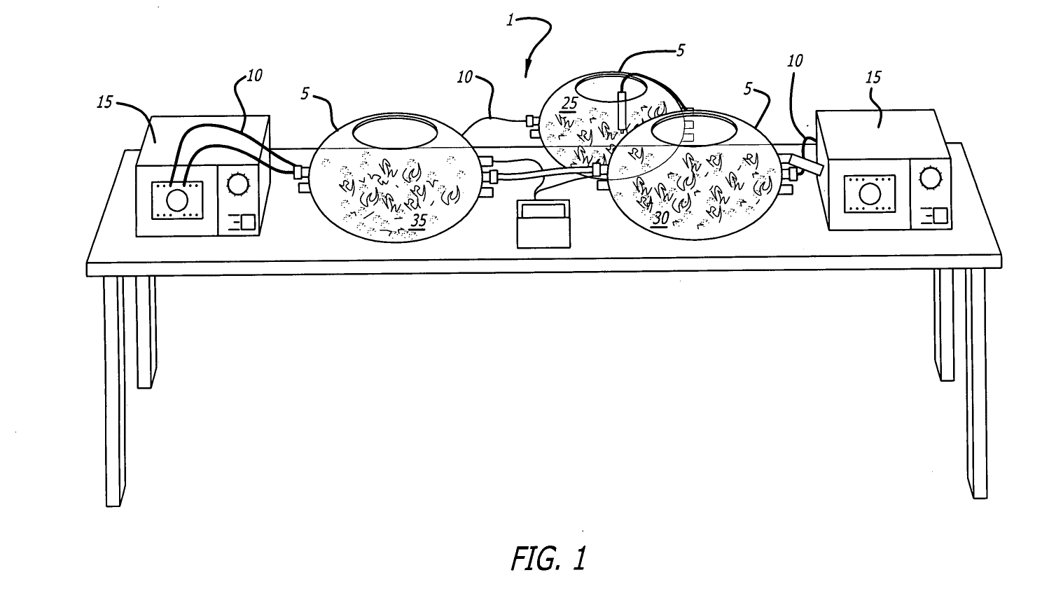

### RELATED APPLICATIONS

This application claims priority to U.S. provisional patent application Ser. No. 60/447,738 filed Jun. 9, 2003 and is also a continuation-in-part to U.S. application Ser. No. 10/346,816, filed on Jan. 16, 2003. The entire contents of both applications are incorporated herein by reference.

### FIELD OF THE INVENTION

The present invention relates to field of cell biology. More specifically the present invention relates to the filed of cell therapy, specifically stem cell therapy. The present invention provides hybrid stem cells and related methods for their preparation and use. The hybrid stem cells of the present invention are useful in treating diseased and damaged tissues and organs in mammals in need thereof.

### BACKGROUND OF THE INVENTION

Since the first description of the isolation of embryonic stem (ES) cells from human blastocysts, many reports have surfaced regarding the isolation and characterization of both embryonic stem cells and adult stem cells. Bongso, A. et al. (1996), Isolation and culture of inner cell mass cells from human blastocyst, Hum. Reprod. U.S.A. 9, 2110-17.

Stem cells (embryonic and adult) are capable of long-term self-renewal and can give rise to mature cell types with specific morphology and function. Similarly, like embryonic stem (ES) cells, which originate from the inner mass of the blastocyst, the origin of adult stem cells share a common origin.

Typically adult stem cells share at least two characteristics: i) they can make identical copies of themselves for long periods of time (long term self-renewal); and they can give rise to mature cell types that have characteristic morphologies and specialized functions. Stem Cells: Scientific Progress and Future Research Directions, Dept. of Health and Human Services, June 2001; http://www.nih.gov/news/stemcell/scireport.htm. Adult stem cells may lack the pluripotential associated with ES cells, however, at least one report has suggested that adult stem cells show more plasticity than previously recognized. Lagasse, E. et al. (2000), Purified hematopoietic stem cells can differentiate into hepatocytes in vivo, Nat. Med. 6, 1229-34.

Ultimately, to demonstrate plasticity, an adult stem cell should give rise to fully differentiated cells that have mature phenotypes. The adult stem cells should also be fully integrated into their new tissue environment and be capable of specialized tissue functions, which are appropriate for that tissue. Stem Cells: Scientific Progress and Future Research Directions, supra.

The difficulty in studying adult stem cell plasticity is establishing that the adult stem cell arises out of one type of cell, or cell population. To date, the best studied adult stem cells are based on bone marrow and brain cells. However, studies using stem cells derived from the bone marrow (i.e. hematopoietic stem cells), stromal cells and/or endothelial cells) and the brain (i.e. neuroblasts) have their limitations. For example, hematopoietic stem cells from the bone marrow are sorted using a cell sorter, which sorts the cells according to various cell surface markers. This methodology yields highly purified to partially purified cell types. In another example, purification of neuronal stem cells are difficult because these cells are localized to different tissues (i.e. olfactory bulb, hipppocampus and lateral ventricles of mice) and not in one convenient location or organ tissue. Altman, J. and Das, G. D. (1965), Autoradiographic and histological evidence of postnatal hippocampal neurogenesis in rats, J. Compl Neurol., 124, 319-335; Altman, J. (1969), Autoradiographic and histological studies of postnatal neurogenesis. IV. Cell proliferation and migration into the anterior forebrain, with special reference to persisting neurogenesis in the olfactory bulb, J. Compl Neurol., 137, 433-457.

Other candidates of adult stem cells are endothelial progenitor cells, skeletal muscle stem cells, epithelial cell precursors in the skin and digestive system and stem cells in the pancreas and liver. Stem Cells: Scientific Progress and Future Research Directions, supra.

Another type of adult stem cell is derived from germ cells, or primordial sex cells (PSC), residing in the lining of the seminiferous tubules of the testes and lining of the ovaries—the spermatogonia and oogonia, respectively. Spermatogonia produce precursor cells that are involved in meiosis. There are at least two types of spermatogonia, type A and type B, and can be differentiated based on unique characteristics. For example, type A spermatogonia are more spherical with a prominent nucleolus and uniformly scattered euchromatin. Whereas, type B spermatogonia tend to be more irregular in shape and smaller with a lobed nucleus. Guillaume E, et al. (2001), Proteome analysis of rat spermatogonia: reinvestigation of stathmin spatio-temporal expression within the testis, Mol. Reprod. Dev., 60(4):439-45. Chiarini-Garcia, H. and Russell, L. D. (2002), Characterization of mouse spermatogonia by transmission electron microscopy, Reproduction, 123(4):567-77. Thus, unlike other adult stem cells, adult germ cells are easy to locate and distinguished from other interstitial cells.

Similar to other adult somatic stem cells, adult germ cells are diploid (2n). In contrast to other adult somatic stem cells, germ cells (spermatogonia and oogonia) contain a genome that is undamaged and unspoiled. Whereas, somatic cellular DNA is more damaged (i.e. free radicals) due to their age and low rate of replenishment. Further, somatic stem cells finally succumb to the forces of differentiation that create the tissues of the body. Thus, methods comprising a stem cell consisting of undamaged DNA is preferred.

A persistent problem with adult stem cell transplants in vivo is that of immune rejection. Thus, to date, recipient's of stem cells are reliant on donors whose cells will not be rejected by the recipien's immune system.

Therefore, improved methods to provide an adult stem cell which has a high rate of long term self-renewal, while being easy to isolate and purify, and at the same time reduce the associated immune rejection when translocated in vivo or in vitro, will ameliorate existing problems associated with stem cell biology and their use as therapeutics.

### INVENTION SUMMARY

One objective of the present invention is to provide a hybrid stem cell (HSC) comprising an enucleated adult stem cell having a nucleus from a primordial sex cell or an embryonic stem cell.

Optionally, the HSC may comprise an enucleated adult stem cell and primordial sex cell derived from the same animal. Additionally, wherein the adult stem cell and primordial sex cell are derived from the same animal, the animal may optionally be a mammal. In a separate embodiment of the invention, the HSC is biologically active in a post natal animal.

In another embodiment of the invention, the HSC comprises an enucleated adult stem cell having a nucleus from a primordial sex cell. In one embodiment, the primordial sex cell is a spermatogium cell. In another different embodiment, the primordial sex cell is an undifferentiated spermatogonium cell. In yet another embodiment, the primordial sex cell is a differentiated spermatogonium cell. Alternatively, in a another separate embodiment, the primordial sex cell may be an oogonium cell.

In an alternative embodiment of the invention, the HSC comprises an enucleated adult stem cell fused with a primordial sex cell using electrofusion. Optionally, in a different embodiment, the HSC comprises an enucleated adult stem cell fused with a primordial sex cell by a virus-based fusion methodology. Alternatively, the HSC comprises an enucleated adult stem cell fused with a primordial sex cell using chemical fusion. Further, the HSC may optionally comprise an enucleated adult stem cell fused with a primordial sex cell using mechanical-based fusion.

Another embodiment of the present invention provides a method for preparing a modified germ cell comprising: (a) obtaining an adult stem cell from a first donor animal; (b) obtaining a primordial sex cell (PSC) from a second donor animal of the same species as the first donor animal; (c) enucleating the adult stem cell; and (d) fusing the enucleated adult stem cell with the PSC.

In a different embodiment of the invention, a therapeutic composition comprises an enucleated adult stem cell having a nucleus from a primordial sex cell or an embryonic stem cell. Optionally, in another embodiment, the therapeutic composition is used to regenerate diseased or damaged tissues of an animal in need thereof. In a different embodiment, the tissue regenerated by the therapeutic composition is heart tissue. In an alternative embodiment, the therapeutic composition regenerates lung, liver, neural, kidney or somatic muscle tissue.

In yet another embodiment, a fused cell comprises an enucleated adult stem cell and one of a primordial sex cell or embryonic stem cell fused to the enucleated adult stem cell. In a different embodiment a primordial sex cell is fused to the enucleated adult stem cell. Optionally, in another embodiment an embryonic stem cell is fused to the enucleated adult stem cell.

Further embodiments of the present invention include an HSC wherein the enucleated adult stem cell and primordial sex cell are derived from different individuals within the same species.

Alternatively the HSC enucleated adult stem cell and primordial sex cell are derived from the same individual.

Furthermore, HSC of the present invention includes a cell comprising an enucleated adult stem cell and the embryonic stem cell that are derived from the same individual.

### DEFINITION OF SPECIFIC TERMS

The term “primordial sex” cell as used herein means a diploid germ cell and/or a spermatogonia and a oogonia.

The term “spermatogonia” as used herein means a primordial male sex cells that give rise to progenitors of primary spermatocytes.

The term “oogonia” as used herein means a primordial female sex cells that serves as a source of ova.

The term “ovum” as used herein means the female gamete, a haploid unfertilized egg, which is capable of developing into a new animal when fertilized by a spermatozoon.

The term “oocyte” as used herein means a developing egg cell in oogenesis and upon undergoing meiosis forms the ovum.

The term “stem cell” as used herein describes a cell able to regenerate and also to give rise to progenitor cells which ultimately will generate cells developmentally restricted to specific lineages.

The term “bioreactor” as used herein means a specialized chamber to grow, expand, maintain, sustain and mature cells in vitro.

The term “hybrid stem cell” as used herein refers to a stem cell made using an enucleated adult stem cell that has a nucleus transplanted from either a primordial germ cell or an embryonic stem cell. The reader is cautioned that it may be common practice to use the abbreviation “HSC” to mean hematopoietic stem cell. However, as used herein “HSC” is an abbreviation for hybrid stem cell.

### DETAILED DESCRIPTION OF THE INVENTION

This description is not to be taken in a limiting sense, but is made merely for the purpose of illustrating the general principles of the invention. The section titles and overall organization of the present detailed description are for the purpose of convenience only and are not intended to limit the present invention.

The term hybrid stem cell used herein describes a cell comprised of an enucleated adult stem cell having a nucleus from a primordial sex cell or an embryonic stem cell.

The present invention described herein is directed at the preparation and use of hybrid stem cell (HSC) compositions. The HSC compositions are generally prepared by providing an enucleated adult stem cell with the nucleus of either a donor germ cell or stem cell. The HSC possess the surface antigens and receptors from the adult stem cell but has a nucleus from a developmentally younger cell. Consequently, the HSCs of the present invention will be receptive to cytokines, chemokines and other cell signaling agents, yet possess a nucleus free from age related damage. Age related damage includes, but is not limited to nucleic acid free radical damage and telomere shortening.

The HSCs made in accordance with the teachings of the present invention are useful in a wide range of therapeutic applications. For example, and not intended as a limitation, the HSCs of the present invention can be used to replenish stems cells in animals whose natural stem cells have been depleted due to age or ablation therapy such as cancer radiation and chemotherapy. In another non-limiting example the HSCs of the present invention are useful in organ regeneration and tissue repair. In one embodiment of the present invention the HSCs can be used to reinvigorate damaged muscle tissue including dystrophic muscles and muscles damaged by ischemic events such as myocardial infarcts. In another embodiment of the present invention the HSC compositions disclosed herein can be used to ameliorate scarring in animals following a traumatic injury or surgery. In this embodiment the HSCs of the present invention are administered systemically, preferably intravenously, and migrate to the site of the freshly traumatized tissue recruited by circulating cytokines the damaged cells secrete.

In one embodiment the HSCs of the present invention utilize an adult stem cell that is enucleated and then fused to either an embryonic stem cell or a primordial sex cell. In one embodiment the enucleated adult stem cell is fused to a primordial sex cell. The enucleated adult stem cell and primordial sex cell can be derived from the same or different animals. The resulting HSC may be made from any animal or animal combination and translocated into any other animal, preferably the HSC is biologically active in a post natal animal.

In one embodiment of the present invention, the primordial sex cell is a spermatogium cell. The primordial sex cell may be undifferentiated spermatogonium cell or a differentiated spermatogonium cell. Alternatively in a different embodiment of the invention, the primordial sex cell is an oogonium cell.

The enucleated adult stem cell may be fused with the primordial sex cell by various methods known to one skilled in the art. For example, such fusion methods include, but are not limited to electrofusion; virus-based fusion methodology; chemical fusion; and mechanical-based fusion. The aforementioned methods are all well known by those skilled in the art. Therefore, it is not necessary to provide a description of this known methods. Furthermore, the method of fusing the enucleated adult stem cell to the primordial sex cell is not limited to the methods listed above. It would be obvious to one skilled in the art to use other fusion methodologies to obtain the same result.

Alternatively, in a different embodiment of the invention, the HSC may comprise an enucleated adult stem cell fused to an embryonic stem cell. For this particular embodiment, the same fusion methodologies listed above may be utilized to obtain the HSC. Furthermore, the fusion technique is not limited to those methods mentioned above.

Alternative methods of enucleation and nucleation are contemplated within the scope of the present invention including mechanical methods of denucleation and renucleation using microsurgical techniques. While it is envisioned that cell fusion technologies may provide for the most biologically active forms of HSCs, the techniques and methods taught in co-pending U.S. patent application Ser. No. 10/346,816 (the '816 application) are also applicable to the present invention. The entire contents of the '816 application are incorporated herein by reference.

The HSCs made in accordance with the teachings of the present invention may be totipotent, pluripotent, multipotent or bipotent. The HSC is capable of forming at least one type of tissue, more particularly, the HSC is capable of forming at least more than one type of tissue. Once the HSC is established, it can be manipulated by various methods described herein to produce desired characteristics. For example, the hybrid stem can be expanded and maintained in a particular medium.

Preparations of the HSCs can be derived from the same species or they can be derived from different species. Translocation of the HSCs can be into the same species host or a different species host.

Alternatively, the primed HSCs can be used to derive cells for therapeutics to treat abnormal conditions and tissue repair.

In another embodiment of the invention, a therapeutic composition comprises an enucleated adult stem cell having a nucleus from a primordial sex cell or an embryonic stem cell. The therapeutic composition may be used to regenerate diseased or damaged tissues of an animal in need thereof. The diseased or damaged tissues may include such tissues as heart tissue, lung tissue and other bodily tissue.

### EXAMPLES

All the cell types and other materials not described herein are obtained through available sources and/or through standard methods used in the art.

Unless defined otherwise, all technical and scientific terms used herein have the same meaning as commonly understood by one of ordinary skill in the art to which the invention belongs.

All publications mentioned herein are incorporated herein by reference to describe and disclose specific information for which the reference was cited in connection with and are not to be construed as an admission that the invention is not entitled to antedate such disclosures by virtue of prior invention.

Throughout this description, the preferred embodiment and examples shown should be considered as exemplars, rather than as limitations on the present invention.

### Example 1

### Isolating the Primordial Sex Cells (PSCs)

The mammal or animal is anesthetized and the gonads are removed and transected. The primary sex cells (PSCs) are isolated with the aid of a microscope. Alternatively, a biopsy punch of the gonads can also be used and the PSCs isolated with the aid of a microscope. Under the microscope, the PSCs have stem cell morphology (i.e. large, round and smooth) and are mechanically retrieved from the gonads. In particular, the spermatogonia and oogonia, are retrieved from the gonads. In particular, type A and type B spermatogonia are retrieved.

To obtain an ova/ovum, the animal is superovulated, and at least one ovum is retrieved and placed in nutritive media to keep it viable. The ova is held in place using a micropipette and with another micropipette enter the ova until the tip is adjacent to the ova nucleus. Enucleating the ova is possible by applying a small vacuum to the micropipette. Discard the ova (1n) nucleus. Enucleation methods (above) are repeated with the PSCs (i.e spermatogonia and/or oogonia), except this time the nucleus is retained and the cytosol is discarded.

Other methods of enucleation and nucleation are contemplated within the scope of the present invention including other mechanical methods as well as methods utilizing electrical stimuli.

### Example 2

### Isolation and Purification of Type A Spermatogonia

The following is an illustrative example for isolating and purifying Type A Spermatogonia. In step 1, the testis from 6-day-old donor mice (n=8) are removed and place into a petri dish with sterile phosphate-buffered saline (PBS) containing 10% penicillin-streptomycin.

Next, in step 2, the testis are decapsulated under a dissection microscope, and the seminiferous cords/tubule is collected, pooled and placed into a conical centrifuge tube containing a solution of 2 mg/ml of collagenase (Sigma Chemicals, St. Louis, Mo.) and 10 μg/ml DNase I (Sigma Chemicals, St. Louis, Mo.) in Dulbecco modified Eagle medium (DMEM; Specialty Media).

In step 3, the contents, after centrifugation, are incubated at 37° C. for 30 minutes on a shaker with occasional gentle pipetting to dissociate the interstitial Leydig cells from the semiferous tubules.

In step 4, after incubation, the tubules are allowed to settle down to the bottom of the tube and the supernatant, containing the Leydig cells is removed.

In step 5, the digestion and settling step are repeated once.

In step 6, the tubules are washed 2X with DMEM and further digested with 2 mg/ml collagenase, 10 μg/ml Dnase I and 1 mg/ml hyaluronidase type III (Sigma Chemicals, St. Louis, Mo.) for 20-30 minutes in a shaking water bath at 37° C. until the peritubular cells detached from the tubules.

In step 7, the tubules are allowed to settle and the supernatant containing the peritubular cells was discarded.

In step 8, a fourth digestion is performed by adding to the pellet 1 ml of DMEM containing 2 mg/ml collagenase, 10 μg/ml Dnase I and 1 mg/ml hyaluronidase type III until a single cell suspension was obtained. This digestion results in a cell suspension containing Sertoli cells and type A spermatogonia.

In step 9, the cells arre washed twice with DMEM and filtered through a 80-μm nylon mesh (Tetko).

In step 10, in order to isolate the type A spermatogonia from the Sertoli cells, the cell mixture is incubated for 1 hour with a 1:200 dilution of rat anti-mouse antibody that recognizes the extracellular domain of c-kit receptor (clone 2B8; Pharmigen). To isolate type A spermatogonia from an adult it is recommended to use a 1:200 dilution of a rat anti-mouse antibody that recognizes the homophilic adhesion molecule, Ep-CAM (clone G8.8, Develomental Studies Hybridoma Bank, University of Iowa, Iowa City, Iowa; Anderson et al, 1999).

In step 11, cells are incubated for 30 minutes on an Orbitron rotator (Boekel Scientific). The cell suspension is then centrifuged, the supernatant removed and the pellet washed twice with DMEM to remove any excess antibody.

In step 12, the cells are resuspended in 4 ml of culture medium. Then, M-450 magnetic beads, coated with a sheep anti-rat immunoglobulin G (Dynabeads; Dynal), are mixed with the cell suspension at a ratio of 4 beads/target cell for 1 hour at 34° C. on a shaker. The c-kit-positive cells are pulled out of the suspension with a magnet applied to the wall of the centrifuge tube. The c-kit-positive cells (type A spermatogonia) stick to the wall. Type A spermatogonia are collected and resuspended in 5 ml of culture medium.

### Example 3

### Isolation and Purification of Adult Stem Cells

The following is an illustrative example of a procedure of isolating and purifying adult animal stem cells, specifically, multi-potent adult progenitor cells (MAPC'S). First, in step 1, the femurs and tibias are removed from 5-8 week old donors and the bones are placed in HBSS+ (Gibco-BRL 14170161)/2% FBS (Hyclone)/10 mM HEPES buffer (Gibco-BRL 15630080), on ice. The bones should be free of muscle and fatty tissue. The bones are cut just before flushing to eliminate a loss of BMC. Additionally, the bones are kept on ice at all times until process.

Next, in step 2, the tibias and femurs are flushed with a 22 gauge needle using a 3 cc syringe filled with HBSS+ (Gibco-BRL 14170161)/2% FBS (Hyclone)/10 mM HEPES buffer (Gibco-BRL 15630080). (Depending on the number of donors used, it is best to try not to use more than 15 ml of HBSS+ when flushing bones so that all of the sample will fit into one 15 ml conical tube.) The BMC is re-suspended using the 18 gauge needle and 3 cc syringe by flushing the suspension up and down. The suspension is flushed forcefully enough to break up clumps, but not so forcefully that cells are damaged. The sample and the media are kept on ice at all possible times.

In step 3, bone marrow mononuclear cells (BMMNC) are collected by Ficoll-Hypaque separation.

In step 4, v1×10⁵/cm² BMMNC is plated out on fibronectin (FN; Sigma Chemicals, St. Louis, Mo.) coated dishes 10 ng/mL.

In step 5, the MAPC media is created consisting of the following: 60% DMEM-LG (Gibco BRL), 40% MCDB-201 (Sigma Chemicals, St. Louis, Mo.) with 1× insulin-transferrin-selenium (ITS), 1× linoleic-acid-bovine-serum-albumin (LA-BSA), 10⁻⁹M dexamethasone (Sigma Chemicals, St. Louis, Mo.), 10⁻⁴M ascorbic acid 2-phosphate (Sigma Chemicals, St. Louis, Mo.), 100 units of penicillin, 1000 units of streptomycin (Gibco BRL) with 2% fetal calf serum (FCS; Hyclon Laboratories), containing 10 ng/mL hPDGF-BB (R&D Systems), 10 ng/mL mEGF (Sigma Chemicals, St. Louis, Mo.), and 1000 units/mL mLIF (Chemicon).

In step 6, BMMNC cultures are maintained at 5×10³/cm² after 3-4 weeks cells are harvested and depleted of CD45⁺/Terr119⁺ cells using a micromagnetic bead separator (Miltenyi Biotec).

In step 7, the CD45⁻/Terr⁻ (˜20%) is plated at 10 cells per well of a FN treated (10 ng/mL) 96-well dish and expanded at densities of 0.5-1.5×10³/cm². Approximately 1% of the wells yield continuous growing MAPC cultures.

Finally, in step 8, the MAPC's can be characterized by being CD3, Gr-1, Mac-1, CD19, CD34, CD44, CD45, cKit and major histocompatibility (MHC) class-I and class-II negative.

### Example 4

### Enucleation of Adult Stem Cells

The following is an illustrative example for enucleating adult stem cells. First, in step 1, adult stem cells isolated as described in Example 3 above, are grown to a confluency of approximately 1×10⁶ under appropriate growth requirements and medium.

Next, for step 2, to enucleate, cells are trypsinized and resuspended in pre-warmed culture medium (37° C.) containing cytochalasin B at a concentration of 10 μg/ml.

In step 3, the cell suspension is centrifuged at 8,500 rpm for 30 minutes at 37° C.

After centrifugation, in step 4, the karyoplast pellet is removed and the cytoplasts are washed once with culture medium.

In the final step 5, the cytoplasts are stained with the fluorescent DNA dye Hoechst 33528 (Sigma Chemicals, St. Louis, Mo. B1155) to test the efficiency of enucleation

### Example 5

### Hoechst 33528 Staining of Enucleated Adult Stem Cells

The following is an illustrative example of staining enucleated adult stem cells. First, in step 1, adult stem cells are placed in culture medium pre-warmed to 37° C. immediately after enucleation.

In step 2 of the process, Hoechst 33528 is added to culture medium to a final concentration of 5 μg/ml.

Next, in step 3, the cells are mixed well and incubated in a 37° C. water bath for 90 minutes exactly, wherein the cells are mixed every few minutes.

In step 4, after the 90 minute incubation period, the cells are centrifuged down at 300×g for 3 minutes at 4° C. and the pellet is resuspended in pre-chilled (4° C.) HBSS (Gibco-BRL 14170161)/2% FBS (Hyclone)/10 mM HEPES buffer (Gibco-BRL 15630080).

In step 5, the stained cells are kept at 4° C. to minimize leakage of Hoechst dye from cells FACS cells and to determine the percent enucleation compared to control cells that have not been treated with cytochalasin B. Hoechst dye is excited with the UV laser at 350 nm and its fluorescence is measured with a 450/20 BP filter (Hoechst Blue) and a 675 EFLP optical filter (Hoechst Red).

### Example 6

### Creating the HSC

In a culture dish containing nutritive media the enucleated ovum is held in place using one micropipette and with another micropipette the nucleus from the donor cell (primordial sex cell of stem cell) is inserted into the enucleated adult stem cell to form the HSC of the present invention.

Enucleated or nucleated stem cell and/or nucleus donor cell and HSC can be cryo-preserved using techniques well known to those having ordinary skill in the tissue culture arts. The cells thus stored can be thawed and used at a later time.

Alternative methods to renucleate a cell including cell fusion methods, all which are within the scope the present invention.

### Example 6

### HSC Expansion: The Bioreactor Chamber

In one embodiment of the present invention HSC expansion is done using a conventional bioreactor. For example, a bioreactor is provided having at least one chamber, preferably at least two chambers. The chamber is used to grow, expand, maintain, sustain and differentiate the HSCs of the present invention. The chambers can be limited to one, but preferably there are at least two chambers. The chambers are comprised of silicon oxide or glass. However, other materials used to construct similar biological chambers can be used.

The chambers are connected by tubing to each other, and further connected by tubing to various ancillary systems including peristaltic pumps micro-oxygenators, CO₂ reserves and molecular sieve filters. The tubing is comprised of neoprene or other similar made materials for use in biological systems. The tubing can have various diameters from ⅛ of an inch to ⅓ of an inch. However, smaller or greater diameter tubing for similar uses is possible. The different size tubing are accommodated by different size fittings of the chamber(s). The tubing allows flow of fluid media in the chambers comprising of nutrients, further comprising of macro and micromolecules, between the chambers. The flow of the nutrients is driven by two peristaltic pumps; or alternatively by at least one pump with multiple heads. Each peristaltic pump or each head of a multi-head peristaltic pump drives fluid flow in one direction. However, using at least two pumps allows for bidirectional fluid flow into and out of the chambers.

Also a pH sensor and pH meter are used to control acid/base balance. The ph sensor is first connected to a ph meter which is secondly immersed below the surface of the media in the chamber. The pH sensor detects drops and rises in pH in the media in the chamber, and will send a stimulus to the pH meter. The pH meter in turn contains wires connected to CO₂ valves further connected by fittings on the chambers. For example, when the pH of the media in the chambers is low, a stimulus back to the pH meter to open the CO₂ valve(s), thereby allowing CO₂ from the CO₂ reserve to flow into the chamber.

Ancillary systems include a CO₂ reserve which supplies CO₂ via the CO₂ valve. Also used is a micro-oxygenator (Aqua Pro) and pump. The micro-oxygenator is connected similar to the CO₂ reserve via a valve and tubing. Fluid from the tubing flows through the micro-oxygenator and is oxygenated by side ports or inlets which inject oxygen into the space; thereby aerating the fluid for improved viability of the cells.

Also a molecular dialysis filter similar to the micro-oxygenator and attachment, fluid flows through the filter and particular sized molecules are restricted, for example, molecules at least about 60 KDa are restricted from the fluid. The dialysis filter works on counter-current system and uni-directional current system.

In addition, highly purified water (i.e., ionized, UV treated and microfiltered) is used to sustain the proper water content in the system. The highly purified water can be added to the media in the chambers by any sterile means available.

The media used in the chambers is any standard cell culture media suitable for supporting the growth of primary cells. For example, a nutritive media comprising at least M15:high glucose DMEM, about 15-20% fetal bovine serum (FBS), 1× 1-glutamine, 1× penicillin/streptomycin, 1× non-essential amino acids, and other growth factors as known to those having ordinary skill in the art of cell biology and cell culture techniques.

### Example 7

### Screening the HSCs for Surface Receptor Expression

The HSCs of the present invention are screened for surface receptors and antigen expression as follows. Cells are removed from the bioreactor after a suitable expansion period has elapsed. A suitable expansion period is defined as at least one population doubling.

Fluorescence Resonance Energy Transfer (FRET) and Bioluminescence Resonance Energy Transfer (BRET) are technologies based on Resonance Energy Transfer (RET). It has been reported that energy transfer efficiency is highly dependent on the distance between the donor and acceptor moieties and their relative orientation with respect to each other. In most RET-based assays, the typical effective distance between the donor and acceptor is 10 to 100 angstroms and this range correlates with most biological interactions. (BRET; Packard BioScience, BioSignal Packard Inc., Meriden, Conn.). The use of BRET and FRET technologies, screen for HSCs with certain numbers of receptors and their location on the cell. Visual identification of receptors using BRET and FRET can be viewed on a larger screen or monitor. These projection systems are standard in the art.

Alternatively, other methods to screen for receptors sites in contemplated within the present invention, although not described herein.

Ultimately, the HSCs will have developed all, or nearly all, or mostly all the receptor sites as that observed on the mature stem cell.

### Example 8

### Translocation into the Recipient in Need Thereof

As previously discussed, there are numerous used for the HSCs of the present invention. For example, patients having suffered an ischemic event such as myocardial infarct have regions of the myocardium that are no longer viable. The damaged myocardium eventually replaces the dead cardiac muscle cells with fibrous scare tissue that not only lack contractile function, but resists contraction. As a result, the patent's heart becomes increasing less efficient and loses its ability to pump sufficient qualities of blood to the body's tissues. Eventually, congestive heart failure occurs and the patient dies. Recently, cell therapy techniques have been applied to treating congestive heart failure by injecting hematopoietic stem cells, skeletal myoblasts (see for example U.S. Pat. Nos. 6,579,523 and 6,682,730 the entire contents of which are incorporated herein by reference, specifically column 14, line 7 through column 18 line 45 of both patents) and mesenchymal stem cells (see for example U.S. Pat. No. 6,387,639, the entire contents of which are incorporated herein by reference, specifically Example 1) directly into, or near the damaged myocardium. Other methods suitable for providing the HSC of the present invention into the heart in need thereof includes transluminal catheters specifically designed to, or adapted to, penetrate into the heart chambers, such as those disclosed in U.S. Pat. No. 6,544,230 (the entire contents of which are incorporated herein by reference) and the like.

In one embodiment the HSCs of the present invention are used to restore or improve contractile function to a damaged region of the myocardium. The HSCs made in accordance with the teachings of the present invention can be administered to the myocardium by direct injection using an injection catheter, or can be administered into one or more coronary artery and allowed to migrate to the damaged tissue. In another embodiment the HSCs are administered into the adventitial tissue of a coronary artery.

In another embodiment of the present invention HSCs are injected systemically into the circulatory system of the host in vivo. In this embodiment the HSCs migrate to regions of damaged tissue such as the liver, lungs and brain. Moreover, the HSCs of the present invention can be administered systemically following a traumatic injury or surgery. The presence of the revitalized HSCs of the present invention will result in rapid healing and minimal scarring.

Again, while the specification describes particular embodiments of the present invention, those of ordinary skill can devise variations of the present invention without departing from the inventive concept.

1. A hybrid stem cell (HSC) comprising:
an enucleated adult stem cell having a nucleus from a primordial sex cell or an embryonic stem cell.

2. The HSC of claim 1 wherein the enucleated adult stem cell and primordial sex cell are derived from the same animal.

3. The HSC of claim 2 wherein the animal is a mammal.

4. The HSC of claim 1 wherein the enucleated adult stem cell and the embryonic stem cell are derived from the same animal.

5. The HSC of claim 1 wherein the enucleated adult stem cell has a nucleus from a primordial sex cell.

6. The HSC of claim 5 wherein the primordial sex cell is a spermatogonium cell.

7. The HSC of claim 5 wherein the primordial sex cell is an undifferentiated spermatogonium cell.

8. The HSC of claim 5 wherein the primordial sex cell is a differentiated spermatogonium cell.

9. The HSC of claim 5 wherein the primordial sex cell is an oogonium cell.

10. The HSC of claim 1 wherein the enucleated adult stem cell is fused with the primordial sex cell using electrofusion.

11. The HSC of claim 1 wherein the enucleated adult stem cell is fused with the primordial sex cell by a virus-based fusion methodology.

12. The HSC of claim 1 wherein the enucleated adult stem cell is fused with the primordial sex using chemical fusion.

13. The HSC of claim 1 wherein the enucleated adult stem cell is fused with the primordial sex cell using mechanical-based fusion.

14. The HSC of claim 1 wherein the HSC is biologically active in a post natal animal

15. A therapeutic composition comprising an enucleated adult stem cell having a nucleus from a primordial sex cell or an embryonic stem cell.

16. The therapeutic composition of claim 15 wherein the therapeutic composition is used to regenerate diseased or damaged tissues of an animal in need thereof.

17. The therapeutic composition of claim 15 wherein the diseased tissue is heart tissue.

18. The therapeutic composition of claim 15 wherein the diseased tissue is lung tissue.

19. A fused stem cell comprising:
an enucleated adult stem cell; and
one of a primordial sex cell or embryonic stem cell fused to the enucleated adult stem cell.

20. The fused stem cell comprising of claim 19 wherein the primordial sex cell is fused to the enucleated adult stem cell.

21. The fused stem cell of claim 20 wherein the enucleated adult stem cell is fused with the primordial sex cell using electrofusion.

22. The fused stem cell of claim 20 wherein the enucleated adult stem cell is fused with the primordial sex cell by a virus-based fusion methodology.

23. The fused stem cell of claim 20 wherein the enucleated adult stem cell is fused with the primordial sex using chemical fusion.

24. The HSC of claim 20 wherein the enucleated adult stem cell is fused with the primordial sex cell using mechanical-based fusion.

25. The fused stem cell of claim 29 wherein the embryonic stem cell is fused to the enucleated adult stem cell.

26. A hybrid stem cell (HSC) comprising:
an enucleated adult stem cell having a nucleus from a primordial sex cell.

27. The HSC of claim 26 wherein the enucleated adult stem cell and primordial sex cell are derived from the same animal.

28. The HSC of claim 27 wherein the animal is a mammal.

29. The HSC of claim 26 wherein the primordial sex cell is a spermatogonium cell.

30. The HSC of claim 26 wherein the primordial sex cell is an undifferentiated spermatogonium cell.

31. The HSC of claim 26 wherein the primordial sex cell is a differentiated spermatogonium cell.

32. The HSC of claim 26 wherein the enucleated adult stem cell and primordial sex cell are derived from different individuals within the same species.

32. The HSC of claim 26 wherein the enucleated adult stem cell and primordial sex cell are derived from the same individual.

33. The HSC of claim 26 wherein the primordial sex cell is an oogonium cell A hybrid stem cell (HSC) comprising:

34. An enucleated adult stem cell having a nucleus from an embryonic stem cell.

35. The HSC of claim 34 wherein the enucleated adult stem cell and embryonic stem cell are from the same species.

36. The HSC of claim 34 wherein the animal is a mammal.

37. HSC of claim 34 wherein the enucleated adult stem cell and the embryonic stem cell are derived from the same individual.
